(19) United States
(12) Patent Application Publication
(10) Pub. No.: US 2026/0286254 A1
(43) Pub. Date: Sep. 24, 2026

(54) Bleach Catalyst and Composition Comprising Said Catalyst

(71) Applicant: Reckitt Benckiser Vanish B.V., Hoofddorp (NL)

(72) Inventors: Andre Chieffi, Milano (IT); Torsten Roth, Mannheim (DE); Federico Spolaore, Milano (IT)

(73) Assignee: Reckitt Benckiser Vanish B.V., Hoofddorp (NL)

(21) Appl. No.: 19/168,171

(22) PCT Filed: Apr. 2, 2024
(86) PCT No.: PCT/EP2024/058923

§ 371 Date: Sep. 23, 2025

(30) Foreign Application Priority Data
Mar. 30, 2023 (GB) 2304725.1

(51) Int. Cl. C11D 3/39; C07D 275/06; C07D 417/04; C11D 3/32; C11D 3/34

(52) U.S. Cl. CPC C11D 3/392; C07D 275/06; C07D 417/04; C11D 3/32; C11D 3/349; C11D 3/3942

(57) ABSTRACT

The present invention relates to a bleach catalyst and to a cleaning composition comprising the bleach catalyst, at least one bleaching agent and at least one bleach activator. The invention also relates to the use of the cleaning composition for the removal of stains and/or the removal of malodour from a garment. It also relates to a method of washing clothes, which comprises providing the composition according to the invention to a laundry washing machine and performing the washing at a temperature lower than 60° C. and for a time of less than 120 minutes.

The present invention relates to a bleach catalyst, to a cleaning composition comprising said catalyst, to the use of the composition for the removal of stains and/or for the removal of malodour from a garment, and to a method of washing clothes.

### BACKGROUND

Bleach catalysts are commonly used to catalyse the oxidation of oxidable compounds on, for example, surfaces such as fabrics, dishes, toilets, countertops, and the like. For example, bleach catalysts are used to catalyse the bleaching action of hydrogen peroxide or of compounds that release hydrogen peroxide, such as peroxy compounds, against various stains.

In the 20^{th} century metal-containing catalysts containing macrocyclic ligands were described for use in bleaching compositions. However, these metal-containing bleach catalysts, in particular manganese-containing catalysts are known to damage textile fabric and have the ability to locally stain or discolour substrates. An alternative to this type of metal-containing catalysts was proposed in Patent Application WO98/39098 to the University of Kansas, which describes catalytic systems and methods for oxidising materials in the presence of catalysts which are complexes of transition metals such as Mn, Fe, or Cr, with selected marcopolycyclic rigid ligands, such a cross-bridged macropolycyclic ligands.

Another type of catalysts was proposed in European Patent Application EP0453003 to Unilever NV, in which it is described that certain types of sulfonimines can be used as bleach catalysts. A particularly preferred sulfonimine described in this document is 3-methyl-1,2-benzisothiazole-1,1-dioxide, which is a cyclic sulfonimine.

One drawback of certain sulfonimines, such as 3-methyl-1,2-benzisothiazole-1,1-dioxide, is that these tend to decompose during storage in solid form. Apart from losing their activity they are also prone to decolouration. This problem has been addressed in Patent Application US 2018/0187130 to Weylchem Wiesbaden GmbH by providing a granulate of the sulfonimines with acids in solid form.

When considering laundry items, consumers associate desirability and wearability of a garment with characteristics such as e.g., whiteness, freshness, and/or absence of stains. It is preferred when these characteristics are achieved in a simple way.

Stains containing polyphenols are particularly difficult to remove with common laundry detergents, chlorine bleaches, existing laundry additives or any home remedies. Example of polyphenol-containing foods are tea, red wine, curry, and chocolate. These compounds have been used as dyes and for tanning garments, which gives an idea of how complicated it is to remove polyphenol-containing stains from a garment.

Curcumin is a hydrophobic polyphenol derived from the rhizome of turmeric (Curcuma longa). Turmeric is a strong natural colorant and the additional presence of oil, fat, meat and other protein components like egg and milk make these types of stains extremely complicated to clean. This means that in cuisines where curry/turmeric plays an important role, people are left with no solution and they have no choice than to cope with curry/turmeric stains residues on their garments, even after washing.

In this sense, normally, people are required to follow long cleaning procedures, involving pre-treatment, vigorous rubbing action, or washings at high temperatures to achieve acceptable stain and malodour removal. For example, washing operations are normally performed at temperatures between 40° C. and 60° C.

Hence, there is a need for a bleach catalyst, which does not decompose during storage. In addition, there is a need for a bleach catalyst, which allows for the removal of stains, in particular stains comprising a polyphenolic compound such as turmeric and/or curry stains.

It is an object of the present invention to provide a bleach catalyst, which does not decompose during storage and provides good catalytic activity.

It is a further object of the present invention to provide a cleaning composition, which can be used to clean hard and/or soft surfaces, in particular soft surfaces such as textiles.

The composition is in particular effective at removing stains, such as polyphenol-containing stains (e.g. turmeric and/or curry stains).

It is a further object of the present invention to provide a method of washing clothes at temperatures lower than 60° C. and preferably at short washing cycles, for example, less than 120 minutes.

### SUMMARY OF THE INVENTION

In a first aspect, the present invention relates to a bleach catalyst having formula (1)

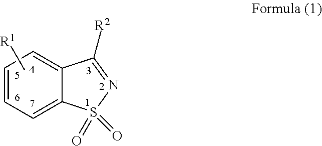

wherein:
   R¹ is H, halogen, hydroxy, amino, nitro, cyano, carboxy, alkyl, C₁-C₁₀-alkoxy, C₁-C₁₀-alkylamino, phenyl, substituted phenyl, or a heterocyclic ring;
   R² is an organic group in which the carbon atom that is bonded to the third carbon of the bleach catalyst is not bonded to any hydrogen atom, and
   the C log P of the bleach catalyst is lower than 2.

In an embodiment, R¹ is H.

In another embodiment R² is:
a branched alkyl or heteroalkyl having 4 to 16 carbon atoms, preferably 4 to 12 carbon atoms, more preferably 4 to 10 carbon atoms, even more preferably 4 to 8 carbon atoms;
a cycloalkyl-, cycloalkenyl-, cycloalkynyl-, or aryl-substituted linear or branched alkyl having 2 to 16 carbon atoms in the alkyl chain, preferably having 2 to 12 carbon atoms in the alkyl chain, more preferably having 2 to 10 carbon atoms in the alkyl chain, even more preferably 2 to 8 carbon atoms in the alkyl chain;
a branched or cyclic alkenyl having 3 to 16 carbon atoms, preferably having 3 to 12 carbon atoms, more preferably having 3 to 10 carbon atoms, even more preferably having 3 to 8 carbon atoms;
a heterocyclic alkenyl;
a cycloalkyl-, cycloalkenyl-, cycloalkynyl-, or aryl-substituted linear or branched alkenyl having 2 to 16 carbon atoms in the alkenyl chain, preferably having 2 to 12 carbon atoms in the alkenyl chain, more preferably having 2 to 10 carbon atoms in the alkenyl chain, even more preferably having 2 to 8 carbon atoms in the alkenyl chain;
a linear alkynyl having 2 to 16 carbon atoms, preferably having 2 to 12 carbon atoms, more preferably having 2 to 10 carbon atoms, even more preferably having 2 to 8 carbon atoms;
a branched alkynyl having 5 to 16 carbon atoms, preferably having 5 to 14 carbon atoms, more preferably having 5 to 12 carbon atoms, even more preferably having 5 to 10 carbon atoms;
a cycloalkyl-, cycloalkenyl-, cycloalkynyl-, or aryl-substituted linear or branched alkynyl having 2 to 16 carbon atoms in the alkynyl chain, preferably having 2 to 12 carbon atoms in the alkynyl chain, more preferably having 2 to 10 carbon atoms in the alkynyl chain, even more preferably having 2 to 8 carbon atoms in the alkynyl chain;
a substituted aryl,
an aryl with multiple or fused rings in which at least one ring is aromatic, or
a heteroaryl.

In an embodiment, R² is:
a branched alkyl or heteroalkyl having 4 to 16 carbon atoms, preferably 4 to 12 carbon atoms, more preferably 4 to 10 carbon atoms, even more preferably 4 to 8 carbon atoms, or
a cycloalkyl-, cycloalkenyl-, cycloalkynyl-, or aryl-substituted linear or branched alkyl having 2 to 16 carbon atoms in the alkyl chain, preferably having 2 to 12 carbon atoms in the alkyl chain, more preferably having 2 to 10 carbon atoms in the alkyl chain, even more preferably 2 to 8 carbon atoms in the alkyl chain;
then the carbon in R² that is bonded to the third carbon atom of the bleach catalyst having formula (1) is a quaternary carbon atom.

In a preferred embodiment, R² is:
a heterocyclic alkenyl,
a substituted aryl,
an aryl with multiple or fused rings in which at least one ring is aromatic, or
a heteroaryl.

In another embodiment, wherein R² is a heterocyclic alkenyl or a heteroaryl.

In a preferred embodiment, R² is pyridyl, quinolyl, isoquinolinyl, acridinyl, pyrazinyl, quinoxalinyl, imidazolyl, benzimidazolyl, purinyl, pyrazolyl, indazolyl, pyrimidinyl, quinazolinyl, pyridazinyl, or cinnolinyl.

In another preferred embodiment, R² is a pyridyl, preferably 2-pyridyl or 3-pyridyl.

In an embodiment, the C log P of the bleach catalyst is lower than 1.8, preferably lower than 1.5, more preferably lower than 1, even more preferably between 0.2 and 0.90, most preferably between 0.3 and 0.85.

In a second aspect, the invention relates to a cleaning composition comprising the bleach catalyst according to the first aspect of the invention, at least one bleaching agent and at least one bleach activator.

In an embodiment, the composition is a hard-surface cleaning composition or a soft-surface cleaning composition, preferably an automatic dishwashing composition or a laundry composition, more preferably a laundry detergent composition or a laundry additive composition, most preferably a laundry additive composition.

In a preferred embodiment, the composition comprises between 0.001 wt. % and 10 wt. % of the bleach catalyst, based on the total weight of the composition, preferably between 0.005 wt. % and 5 wt. %, more preferably between 0.01 wt. % and 1 wt. %, most preferably between 0.025 wt. % and 0.5 wt. %.

In an embodiment, the at least one bleaching agent comprises at least one source of active oxygen selected from the group consisting of an inorganic peroxide, an organic peracid, hydrogen peroxide, and combinations thereof; preferably selected from the group consisting of sodium percarbonate, sodium perborate, ε-phthalimidoperoxy-hexanoic acid (PAP), peracetic acid, potassium peroxymonosulfate (KMPS), and combinations thereof; most preferably wherein said at least one bleaching agent is sodium percarbonate.

In another embodiment, the at least one bleach activator is selected from the group consisting of tetraacetylethylenediamine (TAED), acetylated triazine derivatives, acetylated glycoluriles, acylimides, acetylated glucose, acetylated glycerol, acetylated phenolsulfonates, acetylated phenol carbonic acids, sodium nonanoyloxybenzenesulfonate (NOBS), 4-decanoyloxybenzoic Acid (DOBA), carbonic acid anhydrides, acetylated sugar derivatives, N-acetylated lactams, and combinations thereof; preferably said at least one bleach activator is tetraacetylethylenediamine (TAED).

In a third aspect, the invention relates to a use of the cleaning composition according to the second aspect of the invention for the removal of stains, preferably stains comprising a polyphenolic compound, more preferably wherein the stains comprise turmeric and/or curry stains and/or for the removal of malodour from a garment.

In an embodiment, for the removal of stains from a garment, wherein the garment is made of cotton, synthetic materials or a combination thereof, preferably wherein said synthetic material is polyester.

In a fourth aspect, the invention relates to a method of washing clothes, wherein the method comprises providing the composition according to the second aspect of the invention to a laundry washing machine and performing the washing:
at a temperature lower than 60° C., preferably lower than 40° C., more preferably lower than 30° C., even more preferably lower than 25° C.; and
for a time of less than 120 minutes, preferably less than 60 minutes, more preferably less than 45 minutes, even more preferably less than 40 minutes, most preferably less than 30 minutes

### Definitions

The following definitions are used in the present application.

“alkyl” as used in the present application means a saturated aliphatic radical, such as straight or branched chain group having 1 to 16 carbon atoms, cycloalkyl groups having 3 to 16 carbon groups in their ring, alkyl substituted cycloalkyl groups, and cycloalkyl substituted alkyl groups. Non-limiting examples of straight and branched chain groups are methyl, ethyl, butyl, isopropyl, and 2-ethylhexyl.

“heteroalkyl” as used in the present application means an alkyl group in which one or more atoms is a heteroatom, e.g., oxygen, nitrogen, sulphur, etc. Non-limiting examples of heteroalkyl groups are alkoxy, alkyl-substituted amino, and piperidinyl.

“aryl” as used in the present application means aromatic hydrocarbon radical, which can be substituted, have a single ring, multiple rings or fused rings in which at least one is aromatic. A non-limiting example of an aromatic single ring is phenyl. A non-limiting example of multiple rings is biphenyl. A non-limiting example of multiple fused rings in which at least one is aromatic is phenantryl.

When talking about an aryl with multiple rings or fused rings in which at least one is aromatic, it will be understood that these are polycyclic aryls.

“heteroaryl” as used in the present application means an aryl group in which one or more atoms is a heteroatom, e.g., oxygen, nitrogen, sulphur, etc. It will be understood that a radical having a heteroaryl with one or more fused rings is also considered a heteroaryl. Non-limiting examples of heteroaryl groups are pyridyl, pyrazinyl, pyrimidinyl, pyridazinyl, pyrrolyl, furanyl, thiophenyl, quinolyl, isoquinolinyl, acridinyl, quinoxalinyl, purinyl, indazolyl, quinazolinyl, and cinnolinyl.

“alkaryl” as used in the present application means an aryl group with a saturated straight or branched chain having 1 to 16 carbon atoms; for example, ethylphenyl and decylphenyl.

“alkenyl” as used in the present application means any radical having at least one ethylenic double bond, including straight or branched chain groups having 2 to 16 carbon atoms and at least one double bond, cycloaliphatic alkenyl groups, and aryl substituted alkenyl groups. Non-limiting examples of alkenyl groups are octenyl and methyloctenyl.

“cycloalkenyl” as used in the present application means cyclic species with at least one ethylenic double bond in the ring, excluding aromatic rings. The ring can have between 3 to 16 atoms. These species are also known as cyclic olefins. Non-limiting examples of cycloalkenyl or cyclic olefins are dicyclopentadiene, bicyclo compounds, and oxabicyclo compounds.

“heterocyclic ring” as used in the present application means a radical having a cyclic or ring structure in which one or more atoms is a heteroatom, e.g., oxygen nitrogen, sulphur, etc. Non-limiting examples of heterocyclic rings are heterocyclic alkenyls.

“heterocyclic alkenyl” as used in the present application means a radical having at least one cycloalkenyl in which one or more atoms is a heteroatom, e.g., oxygen nitrogen, sulphur, etc. It will be understood that a radical having a heterocyclic alkenyl with one or more fused rings is also considered a heterocyclic alkenyl. Non-limiting examples of heterocyclic alkenyls are imidazolyl, pyrazolyl, and indazolyl.

“alkynyl” as used in the present application means any radical having at least one triple bond, including straight or branched chain groups having 2 to 16 carbon atoms and at least one triple bond, cycloaliphatic alkynyl groups, and aryl substituted alkynyl groups. Non-limiting examples of alkenyl groups is butynyl.

“cycloalkynyl” as used in the present application means cyclic species with at least one triple bond in the ring. The ring can have between 5 to 16 atoms.

“halogen” as used in the present application means F, Cl, Br, or I.

“organic group” as used in the present patent application means a chemical group or radical that contains carbon-hydrogen or carbon-carbon bonds.

“pyridyl” as used in the present application means any of the three isomeric radicals derived from pyridine.

“quinolyl” as used in the present application means a radical from quinoline.

“isoquinolinyl” as used in the present application means a radical derived from isoquinoline.

“acridinyl” as used in the present application means a radical derived from acridine.

“pyrazinyl” as used in the present application means a radical derived from pyrazine.

“quinoxalinyl” as used in the present application means a radical derived from quinoxaline.

“imidazolyl” as used in the present application means a radical derived from imidazole.

“benzimidazolyl” as used in the present application means a radical derived from benzimidazole.

“purinyl” as used in the present application means a radical derived from purine.

“pyrazolyl” as used in the present application means a radical derived from pyrazole.

“indazolyl” as used in the present application means a radical derived from indazole.

“pyrimidinyl” as used in the present application means a radical derived from pyrimidine.

“quinazolinyl” as used in the present application means a radical derived from Quinazoline.

“pyridazinyl” as used in the present application means a radical derived from pyridazine.

“cinnolinyl” as used in the present application means a radical derived from cinnoline.

“C log P” as used in the present application means logarithm of the partition coefficient of a compound defined as the ratio of concentrations of a compound in a mixture of two immiscible solvents at equilibrium, here n-octanol and water; i.e. C log P=log₁₀(partition coefficient); wherein partition coefficient

$$( P ) = \frac{{[ compound ]}_{n - octanol}}{{[ compound ]}_{water}} .$$

It is calculated using ChemDraw 12.0 (ChemOffice suite by Perkin Elmer), which uses a dataset from full compounds, or fragments, which are experimentally determined, and then modelled in small fragments. Fragment contributions are then added up, with correction factors.

“Laundry detergent composition” as used in the present application means a composition used primarily for the cleaning of garments and whose main action is the removal of grime and dirt.

“Laundry additive composition” as used in the present application means a composition used for providing a specific consumer benefit, such as: removal of stains, elimination of malodour, improvement of whiteness and/or disinfection of garments. This means that the mean action of a laundry additive is not the removal or grime and dirt. Furthermore, a laundry additive is commonly used as a pre-treatment, in soaking conditions or together with a laundry detergent composition.

### DETAILED DESCRIPTION OF THE INVENTION

In a first aspect, the present invention relates to a bleach catalyst having formula (1):

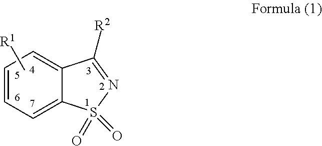

wherein:
R¹ is H, halogen, hydroxy, amino, nitro, cyano, carboxy, alkyl, C₁-C₁₀-alkoxy, C₁-C₁₀-alkylamino, phenyl, substituted phenyl, or a heterocyclic ring;
R² is an organic group in which the carbon atom that is bonded to the third carbon of the bleach catalyst is not bonded to any hydrogen atom, and
the C log P of the bleach catalyst is lower than 2.

The present inventors have observed that when the carbon atom that his bonded to the third carbon atom of the bleach catalyst having Formula (1) is not bonded to any hydrogen atom, then the bleach catalyst is stable against degradation. In other words, when the carbon atom that is bonded to the cyclic sulfone imine in the bleach catalyst having formula (1) is not bonded to any hydrogen atom, then the bleach catalyst is stable against degradation. The inventors have found that the presence of a proton (hydrogen atom) in the carbon atom of the R² group that is attached to the C═N bond leads to undesired yellowing of the catalyst, which is a clear sign of degradation of the catalyst.

However, some bleach catalyst that are stable against colouring, might not have a good performance in terms of catalytic activity. The present inventors have observed that when the partition coefficient of the catalyst, i.e., C log P, is lower than 2, this has a better catalytic activity than similar bleach catalysts. The partition coefficient, C log P, represents the hydrophobicity or lipophilicity of a compound.

In a preferred embodiment, R¹ is H.

In another preferred embodiment, R² is:
a branched alkyl or heteroalkyl having 4 to 16 carbon atoms, preferably 4 to 12 carbon atoms, more preferably 4 to 10 carbon atoms, even more preferably 4 to 8 carbon atoms;
a cycloalkyl-, cycloalkenyl-, cycloalkynyl-, or aryl-substituted linear or branched alkyl having 2 to 16 carbon atoms in the alkyl chain, preferably having 2 to 12 carbon atoms in the alkyl chain, more preferably having 2 to 10 carbon atoms in the alkyl chain, even more preferably 2 to 8 carbon atoms in the alkyl chain;
a branched or cyclic alkenyl having 3 to 16 carbon atoms, preferably having 3 to 12 carbon atoms, more preferably having 3 to 10 carbon atoms, even more preferably having 3 to 8 carbon atoms;
a heterocyclic alkenyl;
a cycloalkyl-, cycloalkenyl-, cycloalkynyl-, or aryl-substituted linear or branched alkenyl having 2 to 16 carbon atoms in the alkenyl chain, preferably having 2 to 12 carbon atoms in the alkenyl chain, more preferably having 2 to 10 carbon atoms in the alkenyl chain, even more preferably having 2 to 8 carbon atoms in the alkenyl chain;
a linear alkynyl having 2 to 16 carbon atoms, preferably having 2 to 12 carbon atoms, more preferably having 2 to 10 carbon atoms, even more preferably having 2 to 8 carbon atoms;
a branched alkynyl having 5 to 16 carbon atoms, preferably having 5 to 14 carbon atoms, more preferably having 5 to 12 carbon atoms, even more preferably having 5 to 10 carbon atoms;
a cycloalkyl-, cycloalkenyl-, cycloalkynyl-, or aryl-substituted linear or branched alkynyl having 2 to 16 carbon atoms in the alkynyl chain, preferably having 2 to 12 carbon atoms in the alkynyl chain, more preferably having 2 to 10 carbon atoms in the alkynyl chain, even more preferably having 2 to 8 carbon atoms in the alkynyl chain;
a substituted aryl,
an aryl with multiple or fused rings in which at least one ring is aromatic, or
a heteroaryl.

For example, when R² is:
a branched alkyl or heteroalkyl having 4 to 16 carbon atoms, preferably 4 to 12 carbon atoms, more preferably 4 to 10 carbon atoms, even more preferably 4 to 8 carbon atoms, or
a cycloalkyl-, cycloalkenyl-, cycloalkynyl-, or aryl-substituted linear or branched alkyl having 2 to 16 carbon atoms in the alkyl chain, preferably having 2 to 12 carbon atoms in the alkyl chain, more preferably having 2 to 10 carbon atoms in the alkyl chain, even more preferably 2 to 8 carbon atoms in the alkyl chain; then the carbon in R² that is bonded to the third carbon atom of the bleach catalyst having formula (1) is a quaternary carbon atom.

In a preferred embodiment, R² is:
a heterocyclic alkenyl,
a substituted aryl,
an aryl with multiple or fused rings in which at least one ring is aromatic, or
a heteroaryl;
preferably R² is a heterocyclic alkenyl or a heteroaryl;
more preferably R² is pyridyl, quinolyl, isoquinolinyl, acridinyl, pyrazinyl, quinoxalinyl, imidazolyl, benzimidazolyl, purinyl, pyrazolyl, indazolyl, pyrimidinyl, quinazolinyl, pyridazinyl, or cinnolinyl;
even more preferably R² is a pyridyl;
most preferably R² is 2-pyridyl or 3-pyridyl.

Preferred catalysts are those in which R¹ is Hydrogen and R² is 2-pyridyl or 3-pyridyl. In this case, the catalyst of formula 1 can be represented as follows:

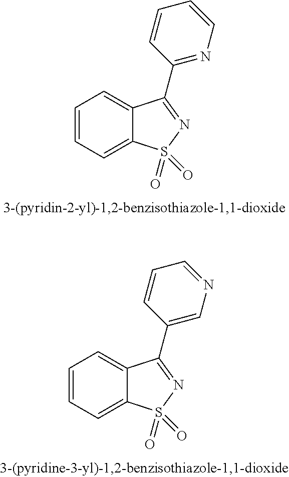 [SMILES: O=S1(=O)N=C(c2ccccn2)c2ccccc21 ; O=S1(=O)N=C(c2cccnc2)c2ccccc21]

In a preferred embodiment, the C log P of the bleach catalyst is lower than 1.8, preferably lower than 1.5, more preferably lower than 1, even more preferably between 0.2 and 0.90, most preferably between 0.3 and 0.85.

### Preparation of Bleach Catalysts According to the Invention

The person skilled in the art will know how to prepare the bleach catalysts according to the invention, for which the reaction mechanism is:

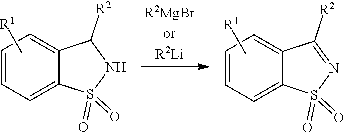

### Preparation of 3-(pyridin-2-yl)-1,2-benzisothiazole-1,1-dioxide

The bleach catalyst 3-(pyridin-2-yl)-1,2-benzisothiazole-1,1-dioxide can be prepared by reacting 1 eq. with a Grignard solution of the pyridine halide, added dropwise under cooling in a water-bath (ca.1.5 h). The suspension is stirred overnight at room temperature. A sample quenched in saturated NH₄Cl shows nearly no starting material. The product is then obtained through recrystallisation from EtOH 1 eq. saccharin with 1 eq. Grignard solution of 2-pyridine halide and the solution is allowed to warm up at room temperature and stirred overnight. Thin layer chromatography analysis showed no starting material and NMR analysis confirmed the purity of the sample.

The setup to obtain the desired molecule is as follow: 30 ml of Methyl tert-butyl ether (MTBE) and then a mixture of 15 ml 2M HCl and 20 ml saturated NaCl is added. The organic phase is separated and washed with 10 ml saturated NaCl. After drying with Na₂SO₄ the solvent is removed and to the residue (1.9 g) 10 ml EtOH are added and removed in vacuo. This is repeated once, and the off-white residue is crystallized from EtOH. The mother liquor contains the impurity contained in the starting material.

### Preparation of 3-(pyridine-3-yl)-1,2-benzisothiazole-1,1-dioxide

Similarly, 3-(pyridine-3-yl)-1,2-benzisothiazole-1,1-dioxide can be prepared by reacting 1 eq. with a Grignard solution of 3-pyridine halide, added dropwise under cooling in a water-bath (ca.1.5 h). The suspension is stirred overnight at room temperature. A sample quenched in saturated NH₄Cl shows nearly no starting material. The product is then obtained through recrystallisation from EtOH eq. saccharin with 1 eq. Grignard solution of 3-pyridine halide. The setup is the same as described above.

In a second aspect, the invention relates to a composition comprising the bleach catalyst according to the first aspect of the invention, at least one bleaching agent and at least one bleach activator.

The composition can be a hard-surface cleaning composition or a soft-surface cleaning composition, preferably an automatic dishwashing composition or a laundry composition, more preferably a laundry detergent composition or a laundry additive composition, most preferably a laundry additive composition.

In an embodiment, the composition comprises between 0.001 wt. % and 10 wt. % of the bleach catalyst, based on the total weight of the composition, preferably between 0.005 wt. % and 5 wt. %, more preferably between 0.01 wt. % and 1 wt. %, even more preferably between 0.025 wt. % and 0.5 wt. %

The at least one bleaching agent may comprise at least one source of active oxygen selected from the group consisting of an inorganic peroxide, an organic peracid, hydrogen peroxide, and combinations thereof; preferably selected from the group consisting of sodium percarbonate, sodium perborate, ε-phthalimidoperoxy-hexanoic acid (PAP), peracetic acid, potassium peroxymonosulfate (KMPS), and combinations thereof; most preferably wherein said at least one bleaching agent is sodium percarbonate.

The composition may then comprise between 1 wt. % and 80 wt. % of the at least one bleaching agent, based on the total weight of the composition, preferably between 5 wt. % and 70 wt. %, more preferably between 10 wt. % and 65 wt. %, even more preferably between 15 wt. % and 60 wt. %. For example, when the cleaning composition is a laundry additive composition, it may comprise between 18 wt. % and 58 wt. % of the at least one bleaching agent.

The at least one bleach activator may be selected from the group consisting of tetraacetylethylenediamine (TAED), acetylated triazine derivatives, acetylated glycoluriles, acylimides, acetylated glucose, acetylated glycerol, acetylated phenolsulfonates, acetylated phenol carbonic acids, sodium nonanoyloxybenzenesulfonate (NOBS), 4-decanoyloxybenzoic Acid (DOBA), carbonic acid anhydrides, acetylated sugar derivatives, N-acetylated lactams, and combinations thereof; preferably said at least one bleach activator is tetraacetylethylenediamine (TAED).

The composition may then comprise between 0.1 wt. % and 20 wt. % of the at least one bleach activator, based on the total weight of the composition, preferably between 0.25 wt. % and 15 wt. %, more preferably between 0.5 wt. % and 10 wt. %, most preferably between 1 wt. % and 5 wt. %.

The composition according to the invention allows for the incorporation of enzymes. For example, the cleaning composition can comprise at least one enzyme selected from the group consisting of a mannanase, a lipase, an amylase, a cellulase, a protease, and combinations thereof. For example, the cleaning composition can comprise between 0.001 wt. % and 10 wt. % of the at least one enzyme, based on the total weight of the composition, preferably between 0.001 wt. % and 8 wt. %, more preferably between 0.001 and 6 wt. %, even more preferably between 0.001 and 4 wt. %, most preferably between 0.001 wt. % and 2 wt. %.

The compositions of the present invention may take any form, e.g., solid, liquid, gel, powder or mixtures thereof. Preferably, the compositions will be in the form of a solid or a powder.

Preferably the composition further comprises a rheology modifier agent, a fragrance, a phase stabilizer, a dye, a softening agent, a chelating agent, an anti-bacterial agent, a transition metal compound, an anti-foaming agent, a preservative, one or more surfactants, one or more fillers, a dye-transfer inhibitor, an optical brightener, or combinations thereof.

The composition may comprise one or more non-ionic surfactants. The amount of said one or more non-ionic surfactants in the composition may be between 0.01 wt. % and 10 wt. %, based on the total weight of the composition, preferably between 0.05 wt. % and 5 wt. %, more preferably between 0.1 wt. % and 1.0 wt. %. Examples of non-ionic surfactants are ethoxylated alcohols and ethoxylated alkyl phenols having the formula R¹(OCH₂CH₂)ₙOH; wherein R¹ is an aliphatic hydrocarbon radicals containing between 10 and 24 carbon atoms or alkyl phenyl radicals in which the alkyl groups contain from 10 to 22 carbon atoms; and n has an average value of from 1 to 10. Preferably, the one or more non-ionic surfactants have the formula R¹(OCH₂CH₂)ₙOH; wherein R¹ is an alkyl moiety having between 10 and 24 carbon atoms, preferably between 12 and 20 carbon atoms, more preferably between 12 and 18 carbon atoms; and n has an average value of from 1 to 10.

For example, when R¹ is defined as being a C₁₂₋₁₄ moiety, it is meant an alkyl moiety having between 12 and 14 carbon atoms. For example, when R¹ is defined as being a C₁₂₋₁₆ moiety, it is meant an alkyl moiety having between 12 and 16 carbon atoms. For example, when R¹ is defined as being a C₁₃ moiety, it is meant an alkyl moiety having 13 carbon atoms.

In a preferred embodiment, the composition comprises between 0.01 wt. % and 10 wt. % of one or more non-ionic surfactants, based on the total weight of the composition, preferably between 0.05 wt. % and 5 wt. %, more preferably between 0.1 wt. % and 1.0 wt. %; wherein said one or more non-ionic surfactants have the formula R¹(OCH₂CH₂)ₙOH; wherein R¹ is an alkyl moiety having between 12 and 16 carbon atoms, and n has an average value of from 3 to 8, preferably between 4 and 6.

The composition may further comprise any conventional anionic surfactant or a mixture of them used in detergent products. These include, for example, alkyl benzene sulfonic acids and their salts as well as alkoxylated or non-alkoxylated alkyl sulphate materials. The anionic surfactants may be present in acid form or in neutralized (e.g., salt) form. The anionic surfactants may be linear, branched, or a mixture thereof. An example of an anionic surfactant is sodium cumenesulphonate.

Examples of anionic surfactants are the alkali metal salts of C₁₀₋₁₆ alkyl benzene sulphonic acids or C₁₁₋₁₄ alkyl benzene sulphonic acids.

Another exemplary type of anionic surfactant is alkoxylated alkyl sulphate surfactants, such as ethoxylated alkyl sulphate surfactants. Such materials are also known as alkyl ether sulphates or alkyl polyethoxylate sulphates.

Alkyl ether sulphates are generally available in the form of mixtures comprising varying R′ chain lengths and varying degrees of ethoxylation. Frequently such mixtures also contain some non-ethoxylated alkyl sulphate (“AS”) materials.

The composition may optionally contain a filler. Suitable fillers include bicarbonates and carbonates of metals, such as alkali metals and alkaline earth metals. Examples include sodium carbonate, sodium bicarbonate, calcium carbonate, calcium bicarbonate, magnesium carbonate, magnesium bicarbonate and sesqui-carbonates of sodium, calcium and/or magnesium. Other examples include metal carboxy glycine and metal glycine carbonate. Chlorides, such as sodium chloride; citrates; and sulphates, such as sodium sulphate, calcium sulphate and magnesium sulphate, may also be employed. Preferably the composition comprises at least one filler selected from the group comprising alkyl carbonate, alkyl sulphate and combinations thereof; more preferably selected from the group comprising sodium carbonate, sodium sulphate and combinations thereof.

When a filler is present, the composition may comprise between 0.1 wt. % and 80 wt. % of said at least one filler, based on the total weight of the composition, preferably between 10 wt. % and 75 wt. %, more preferably between 20 wt. % and 70 wt. %.

The composition may comprise an optical brightener, preferably selected from the group comprising stilbene, benzidine, benzothiazole, benzimidazole, benzoxazole, coumarin, pyrazoline, naphtalimide, naphtoxazole, distyryl-biphenyl, benzonitrile, benzopyrazole, including their derivatives and substituted compounds, and combinations thereof. Suitable optical brighteners include 4,4′-bis(2-sulphostyryl)diphenyl, 7-Diethylamino-4-methylcoumarin, benzoxazole, 2,2′-(1,2-ethenediyl) bis[5-methyl]including their substituted compounds, 4,4′-bis(triazine-2ylamino) stilbene-2,2′-disulphonic acid, mono (azol-2-yl) stilbene and bis(azol-2yl) stilbene; styryl derivatives of benzene and biphenyl, such as 1,4-bis (styryl) benzene, 4,4′-bis (styryl) benzene, 4,4′-bis-(styryl) biphenyl, 4,4′-bis (sulphostryryl) biphenyl sodium salt; pyrazolines such as 1, 3-diphenyl-2-pyrazoline; bis(benzene-2-yl) derivatives, bis(benzoxazol-2-yl) derivatives and bis(benzimidazol-2-yl) derivatives; 2-(benzofuran-2-yl) benzimidazole; coumarins such as 4-methyl-7-hydroxy-coumarin or 4-methyl-7-diethylaminocoumarin; carbostyrils; naphthalimides; dibenzothiophene5,5-dioxide; pyrene; or pyridotriazole derivatives and combinations thereof.

When an optical brightener is present, the composition may comprise between 0.001 wt. % and 1 wt. % of at least one optical brightener, based on the total weight of the composition, preferably between 0.01 wt. % and 0.5 wt. %, more preferably between 0.05 wt. % and 0.25 wt. %.

The composition may further comprise a dye-transfer inhibitor. Preferred dye-transfer inhibitors (sometimes called dye anti-redisposition agents or soil suspending agents) include polyvinylalcohol, fatty amides, sodium carboxylmethyl cellulose, hydroxypropyl methyl cellulose, polyvinylpyrollidone, polyvinylimidazole, polyvinyloxazolidone, polyamine N-oxide polymers and copolymers or N-vinylpyrollidone and N-vinylimidazole.

When a dye-transfer inhibitor is present, the composition may comprise between 0.001 wt. % and 1.5 wt. % of at least one dye transfer inhibitor, based on the total weight of the composition, preferably between 0.01 wt. % and 1.0 wt. %, more preferably between 0.05 wt. % and 0.75 wt. %.

In a third aspect, the invention relates to the use of the cleaning composition of the second aspect of the invention for the removal of stains, preferably stains comprising a polyphenolic compound, more preferably wherein the stains comprise turmeric and/or curry stains and/or for the removal of malodour from a garment.

The composition can be used for the removal of stains from a garment, wherein the garment is made of cotton, synthetic materials, or a combination thereof, preferably wherein said synthetic material is polyester.

In a fourth aspect, the invention relates to a method of washing clothes (or remove stains), wherein the method comprises providing the composition according to the second aspect of the invention to a laundry washing machine and performing the washing at a temperature lower than 60° C. and for a time shorter than 120 minutes.

In one embodiment, the washing in the method is performed a temperature lower than 40° C., preferably lower than 30° C., more preferably lower than 25° C.

In another embodiment, the washing in the method is carried out for a time shorter than 60 minutes, preferably shorter than 45 minutes, more preferably shorter than 40 minutes, even more preferably shorter than 30 minutes.

When carrying out the method according to the fourth aspect preferably between 0.5 g and 20 g of the composition are added to the laundry washing machine per litre of washing liquor, more preferably between 1 g and 15 g, even more preferably between 2 g and 10 g, most preferably between 2.5 g and 7.5 g.

It would be understood that when the clothes are washed in the washing machine, preferably stains and/or malodour are removed.

The foregoing aspects may be freely combined with any of the foregoing aspects disclosed herein.

The invention will be further described, by way of example, with the reference to the following non-limiting embodiments.

### EXAMPLES

### Stability of Bleach Catalysts

The stability of bleach catalysts having the structures shown in Table 1 was determined by preparing solutions of these compounds and measuring their UV-vis absorbance. The person skilled in the art will know how to synthesise the catalysts shown below starting from saccharine.

TABLE 1
Bleach catalysts

| Bleach | | |
|---|---|---|
| catalyst | Name | Structure |
| A | 3-methyl-1,2- benzisothiazole- 1,1-dioxide | 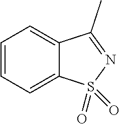 |
| B | 3-phenyl-1,2- benzisothiazole- 1,1-dioxide | 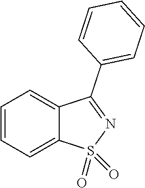 [SMILES: O=S1(=O)N=C(c2ccccc2)c2ccccc21] |
| C | 3-tert-butyl-1,2- benzisothiazole- 1,1-dioxide | 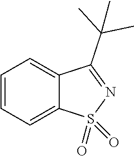 |
| D | 3-lauryl-1,2- benzisothiazole- 1,1- dioxide | 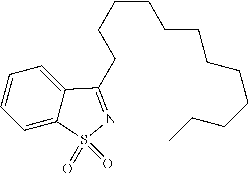 [SMILES: CCCCCCCCCCCCC1=NS(=O)(=O)c2ccccc21] |
| 1 | 3-(pyridin-2-yl)- 1,2- benzisothiazole- 1,1- dioxide | 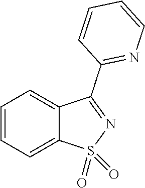 [SMILES: O=S1(=O)N=C(c2ccccn2)c2ccccc21] |
| 2 | 3-(pyridin-3-yl)- 1,2- benzisothiazole- 1,1- dioxide | 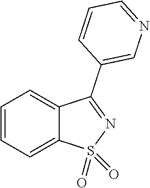 [SMILES: O=S1(=O)N=C(c2cccnc2)c2ccccc21] |

### Samples Preparation

A solution of each compound was prepared by dissolving each compound in a mixture of 50 wt. % water and 50 wt. % acetone. The concentration of each solution was 0.28 mM.

The pH of each solution was set to 11 by adding few drops of a diluted solution of sodium percarbonate to mimic the alkaline conditions of the final formula.

### UV-Vis Absorbance Measurement

The UV-vis absorption of each solution was measured just after preparing the samples (t=0), then at 2 h and 48 h using a UV-Vis Evolution 220 FC-1135 spectrophotometer using a 2 mm quartz cuvette. Table 2 shows the absorption values of each sample at a wavelength of 445 nm and the appearance of the samples under alkaline conditions.

TABLE 2
Absorbance of solutions of bleach catalysts at 445 nm

| | Bleach | Absorption at 445 nm | | Appearance | |
|---|---|---|---|---|---|
| Solution | catalyst | t = 0 | t = 48 h | t = 0 | t = 48 h |
| Solution A | A | <0.05 | 0.37 | Transparent | Yellowing |
| Solution B | B | <0.05 | <0.05 | Transparent | Transparent |
| Solution C | C | <0.05 | <0.05 | Transparent | Transparent |
| Solution D | D | <0.05 | 0.07 | Transparent | Yellowing |
| Solution 1 | 1 | <0.05 | <0.05 | Transparent | Transparent |
| Solution 2 | 2 | <0.05 | <0.05 | Transparent | Transparent |

Table 2 clearly shows that the solutions containing a bleach catalyst in which the carbon atom that is bonded to the carbon atom in position 3 of the bleach catalyst, i.e. bonded to the cyclic sulfone imine, is bonded to a hydrogen atom (bleach catalysts A and D) gets coloured/yellow after some time, for example, after 48 h. This is further evidenced by the UV-Vis absorption measurements, in which the solutions absorb more light at 445 nm after 48 h. This instability of the catalyst is a relevant issue, as performance and sensorial aspects of the product are affected.

### Performance of Bleach Catalysts

### Determination of Partition Coefficient C Log P

Another key aspect of a bleach catalyst is that it must be able to perform. The present inventors have surprisingly observed that the performance of a bleach catalyst is directly linked with its octanol/water partition coefficient (C log P).

This parameter has been determined according to the method shown above, and the C log P value of several bleach catalysts is shown in Table 3.

TABLE 3
Octanol/Water partition coefficient (ClogP) of bleach catalysts

| Bleach catalyst | ClogP |
|---|---|
| A | 0.82 |
| B | 2.25 |
| C | 2.06 |
| D | 6.64 |
| 1 | 0.76 |
| 2 | 0.76 |

### Kinetic of Tropaeolin-O Degradation

These tests were performed following the procedure described in US2012/0205581 A1. Here, Tropaeolin-O was used as sensor molecule to describe the oxidation kinetic of the different catalysts. The catalytic activity of a bleach catalyst is given by the photocatalytic degradation of tropaeolin-O under visible light irradiation.

The kinetic of tropaeolin-O degradation of each sample was determined by measuring the UV-Vis absorbance of the sample every 60 seconds for a total period of 20 minutes at 430 nm. This was done using a UV-Vis Evolution 220 FC-1135 spectrophotometer.

### Samples Preparation for Tropaeolin-O Degradation

A base detergent system containing tropaeolin-O was prepared by adding to 1000 mL of demineralized water at 40° C., the following ingredients:
2.92 g of Ariel Gel (as sold in the UK),
0.088 g of Tropaeolin-O (purity ex. Sigma and used as received),
1.75 g of Sodium percarbonate (purity: 13.3 wt. % active oxygen),
0.10 g of bleach activator tetraacetylethylenediamine (TEAD, purity: 92 wt. % active)

Detergent systems to be tested for the determination of the tropaeolin-O degradation were based on the base detergent system shown above to which a specific bleach catalyst was added, so that the concentration of the bleach catalyst in the detergent system was 0.0144 mM.

TABLE 4
Detergent systems for tropaeolin-O degradation

| Detergent System | Ingredients |
|---|---|
| Base | 2.92 g of Ariel Gel as sold in the UK, |
| | 0.088 g of Tropaeolin-O, |
| | 1.75 g of Sodium percarbonate, |
| | 0.10 g of bleach activator |
| | tetraacetylethylenediamine (TEAD) |
| Detergent system A | Base system + bleach catalyst A |
| | (concentration 0.0144 mM) |
| Detergent system B | Base system + bleach catalyst B |
| | (concentration 0.0144 mM) |
| Detergent system C | Base system + bleach catalyst C |
| | (concentration 0.0144 mM) |
| Detergent system D | Base system + bleach catalyst D |
| | (concentration 0.0144 mM) |
| Detergent system 1 | Base system + bleach catalyst 1 |
| | (concentration 0.0144 mM) |
| Detergent system 2 | Base system + bleach catalyst 2 |
| | (concentration 0.0144 mM) |

### Kinetic of Tropaeolin-O Degradation

The 430 nm absorption peak associated to the decomposition of the sensor molecule, i.e., Tropaeolin-O, after addition of a detergent system was followed over time. Table 5 shows the absorbance of tropaeolin-O at different times at 430 nm. Absorbance in Table 5 is given in absorbance units (a.u.)

TABLE 5
UV-Vis absorption at 430 nm of the tropaeolin-O decomposition
kinetics for the detergent systems in Table 4.

| | | | Absorbance (a.u.) | | |
|---|---|---|---|---|---|
| Detergent System | ClogP | pH | t = 0 min | t = 10 min | t = 20 min |
| Base | — | 10.2 | 1.43 | 1.41 | 1.36 |
| Detergent system A | 0.82 | 10.2 | 1.43 | 0.92 | 0.56 |
| Detergent system B | 2.25 | 10.2 | 1.43 | 1.37 | 1.29 |
| Detergent system C | 2.06 | 10.2 | 1.43 | 1.41 | 1.37 |
| Detergent system D | 2.06 | 10.2 | 1.43 | 1.39 | 1.35 |
| Detergent system 1 | 0.76 | 10.2 | 1.43 | 0.33 | 0.18 |
| Detergent system 2 | 0.76 | 10.2 | 1.43 | 0.60 | 0.22 |

Table 5 shows that detergent systems containing a bleach catalyst with a C log P value lower than 2, clearly have a better performance than those detergent systems containing a bleach catalyst with a C log P higher than 2. This is evidenced as the absorbance values have a faster decrease with time.

Furthermore, the bleach catalysts according to the invention (bleach catalysts 1 and 2) in which:
the carbon atom that is bonded to the third carbon atom of the bleach catalyst, i.e. bonded to the cyclic sulfone imine, is not bonded to any hydrogen bonded, and
the C log P is lower than 2, do not only have an enhanced activity (i.e., faster treopaeolin-O decomposition), they are also stable.

### Tergotometer Test

The bleaching performance for real fabric staining was performed in a Tergotometer (TRG 800i Copley FC-1134) using the following technical stains:
W-10-LI Red Wine
C-S-34 Curry
W-10J Tea
W-10SG Spaghetti Sauce with meat
C-S-67 Mustard
C-S-15 Blueberry Juice

To carry out the tergotometer tests the detergent systems of Table 6 were used.

TABLE 6
Detergent systems for tergotometer test

| Detergent System | Ingredients |
|---|---|
| Ter-Base | 2.92 g of Ariel Gel (as sold in the UK), |
| | 1.75 g of Sodium percarbonate |
| | (13.3 wt. % active oxygen), 0.10 g of |
| | bleach activator tetraacetylethylenediamine |
| | (TEAD, purity: 92 wt. % active) |
| Ter-Det. system A | Base system + bleach catalyst A |
| | (concentration 0.0144 mM) |
| Ter-Det. system B | Base system + bleach catalyst B |
| | (concentration 0.0144 mM) |
| Ter-Det. system C | Base system + bleach catalyst C |
| | (concentration 0.0144 mM) |
| Ter-Det. system 1 | Base system + bleach catalyst 1 |
| | (concentration 0.0144 mM) |
| Ter-Det. system 2 | Base system + bleach catalyst 2 |
| | (concentration 0.0144 mM) |

The tergotometer tests were carried out by dissolving the detergent system of table 6 to be tested in 1 litre of water. After approximately 1 minute the above-mentioned technical stains were added to the washing liquor. Four replicates for each technical stain were carried out, for a total of 24 experiments. Each test was performed at 20° C.±1 and at 40° C.±1, this to show the stain removal properties of each detergent system at two temperatures.

The technical stains were washed with the detergent system of Table 6 to be tested for approximately 30 minutes, in other words, each test lasted 30 minutes. After the technical stains were washed, these were allowed to dry overnight under a blotting paper covering and then ironed. Finally, the stain removal performance of each detergent system was determined using a Datacolor 650-05 spectrophotometer by measuring the Y-value of the technical stain after washing. A higher Y-value means a better stain removal. The results of the tergotometer tests are shown in table 7; these results are provided as the average of the Y-values for bleachable stains at 20° C. and at 40° C. It is worth mentioning that the above-mentioned technical stains are bleachable stains.

TABLE 7
Tergotometer test results using the detergent systems in table
6. Values are given as average Y-values of all replicates.

| | pH tested | Y-value | Y-value |
|---|---|---|---|
| Detergent system | solution | at 20° C. | at 40° C. |
| Ter-Base | 10.2 | 67.6 | 76.1 |
| Ter-Det. system A | 10.2 | 68.8 | 78.1 |
| Ter-Det. system B | 10.2 | 67.7 | 76.5 |
| Ter-Det. system C | 10.2 | 67.8 | 76.5 |
| Ter-Det. system 1 | 10.2 | 68.4 | 77.9 |
| Ter-Det. system 2 | 10.2 | 71.8 | 78.8 |

As can be seen in Table 7, the detergent systems comprising the bleach catalysts according to the invention are particularly effective at removing stains at temperatures not only 40° C. but also at temperatures lower than 20° C.

### Washing Machine Test

The bleaching performance for real fabric staining was performed inside a Bosch washing machine (WAN28268IT), using a “Mix 20° C.” washing protocol, with an estimated total length of washing cycle of about 60 min. The washing machine was loaded with 3.5 kg of ballast to mimic real usage conditions, and four external replicates were performed for each laundry detergent composition. The detergent systems in Table 8 were tested.

TABLE 8
Detergent systems for washing machine tests

| Detergent system | Ingredients |
|---|---|
| Det-WM-0 | 60 g commercial detergent |
| | 35 g laundry bleach composition of table 9 |
| Det-WM-A | 60 g of commercial detergent |
| | 35 g of laundry bleach composition |
| | of table 9 with a 0.0144 mM |
| | concentration of bleach catalyst A. |
| Det-WM-1 | 60 g of commercial detergent |
| | 35 g of laundry bleach composition |
| | of table 9 with a 0.0144 mM |
| | concentration of bleach catalyst 1. |
| Det-WM-2 | 60 g of commercial detergent |
| | 35 g of laundry bleach composition |
| | of table 9 with a 0.0144 mM |
| | concentration of bleach catalyst 2. |

TABLE 9
Laundry bleach composition for use in washing machine tests

| Ingredient | Wt. % |
|---|---|
| Sodium Percarbonate (13.3 wt. % active oxygen) | 30-45 |
| TAED (92 wt. % active) | 2-5 |
| Bleach catalyst | 0.01-0.50 |
| Fillers | 45-60 |
| Surfactants | 0.5-5.0 |
| Enzymes | 0.5-2.0 |
| Dye transfer inhibitor | 0.1-1.0 |
| Additional ingredients | 0.5-2.0 |

In table 9:
The fillers are a combination of sodium carbonate, sodium sulphate, and zeolite,
The enzymes are protease, mannanase, lipase, amylase, cellulase, or combinations thereof,
The additional ingredients are fragrances, optical brighteners, or combinations thereof.

Technical stains were washed in the washing machine using the detergent system of Table 8. Stain removal was determined using a Datacolor 650 spectrophotometer and by measuring the Y-value of the standard soil after washing. A higher Y-value represents a better stain removal. The results are shown in table 10.

TABLE 10
Stain removal performance of detergent systems
in table 8 in a washing machine.

| | | | Y-values - Detergent | | | |
|---|---|---|---|---|---|---|
| | Standard | | system Det-WM- | | | |
| | soil | Description | 0 | A | 1 | 2 |
| Bleach- | C-BC-02 | coffee | 65.71 | 70.01 | 73.59 | 72.80 |
| able | C-S-08 | grass | 80.14 | 79.51 | 80.81 | 80.52 |
| | C-S-12 | blackcurrant | 60.93 | 64.91 | 67.70 | 66.32 |
| | | juice | | | | |
| | C-S-15 | blueberry juice | 67.96 | 70.99 | 74.23 | 73.46 |
| | C-S-67 | mustard | 81.73 | 82.90 | 84.64 | 83.44 |
| | E-164 | grass | 68.78 | 69.81 | 72.68 | 71.72 |
| | W-10J | tea | 71.45 | 72.57 | 75.04 | 73.48 |
| | W-10K | coffee | 83.60 | 85.86 | 87.61 | 87.60 |
| | W-10LI | wine | 69.54 | 69.45 | 73.20 | 72.57 |
| | W-10SG | spaghetti sauce | 58.25 | 58.17 | 58.74 | 59.91 |
| | | with meat | | | | |
| | W-10U | curry | 84.63 | 85.97 | 88.04 | 88.19 |
| | WE5RWWKC | Red wine | 70.14 | 73.39 | 76.66 | 77.49 |
| | | Average | 71.91 | 73.63 | 76.08 | 75.62 |
| Enzy- | C-S-06 | salad | 62.43 | 63.21 | 61.84 | 60.67 |
| matic | | dressing/pigment | | | | |
| | C-S-27 | potato | 66.52 | 67.06 | 64.23 | 63.87 |
| | | starch/colorant | | | | |
| | C-S-406 | Balsamic | 53.15 | 52.97 | 51.85 | 49.95 |
| | | Dressing | | | | |
| | C-S-68 | chocolate ice | 69.22 | 69.72 | 72.53 | 71.15 |
| | | cream | | | | |
| | E-111 | blood | 71.28 | 78.87 | 81.68 | 77.68 |
| | W-10Z | chocolate | 66.23 | 69.98 | 69.73 | 70.04 |
| | | Average | 64.80 | 66.97 | 66.98 | 65.56 |
| Deter- | C-S-17 | make up | 53.99 | 58.59 | 58.69 | 57.49 |
| gency | W-10D | skin | 58.81 | 65.34 | 64.01 | 59.58 |
| | | grease/pigment | | | | |
| | W-10GM | motor oil used | 41.42 | 41.81 | 41.89 | 41.53 |
| | W-10MU | make up | 59.17 | 62.51 | 61.98 | 62.76 |
| | W-10RM | Soot mineral oil, | 32.33 | 36.06 | 35.71 | 34.09 |
| | | IEC | | | | |
| | W-10V | soy sauce | 87.16 | 87.96 | 88.81 | 88.63 |
| | | Average | 55.48 | 58.71 | 58.52 | 57.34 |

(Each value in table 20 is the average of four measurements)

The results shown in Table 10 clearly indicate that the detergent systems comprising the bleaching agent according to the invention have a superior cleaning performance, in particular when considering the removal of bleachable stains.

Hence one or more objects of the present invention are achieved by the present which is further elucidated in the appended claims.

1. A bleach catalyst having formula (1) 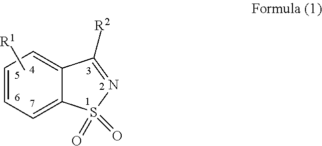
wherein:
R¹ is H, halogen, hydroxy, amino, nitro, cyano, carboxy, alkyl, C₁-C₁₀-alkoxy, C₁-C₁₀-alkylamino, phenyl, substituted phenyl, or a heterocyclic ring;
R² is an organic group in which the carbon atom that is bonded to the third carbon of the bleach catalyst is not bonded to any hydrogen atom, and
the C log P of the bleach catalyst is lower than 2.

2. The bleach catalyst according to claim 1, wherein R¹ is H.

3. The bleach catalyst according to claim 1, wherein R² is:
a branched alkyl or heteroalkyl having 4 to 16 carbon atoms;
a cycloalkyl-, cycloalkenyl-, cycloalkynyl-, or aryl-substituted linear or branched alkyl having 2 to 16 carbon atoms in the alkyl chain;
a branched or cyclic alkenyl having 3 to 16 carbon atoms;
a heterocyclic alkenyl;
a cycloalkyl-, cycloalkenyl-, cycloalkynyl-, or aryl-substituted linear or branched alkenyl having 2 to 16 carbon atoms in the alkenyl chain;
a linear alkynyl having 2 to 16 carbon atoms;
a branched alkynyl having 5 to 16 carbon atoms;
a cycloalkyl-, cycloalkenyl-, cycloalkynyl-, or aryl-substituted linear or branched alkynyl having 2 to 16 carbon atoms in the alkynyl chain;
a substituted aryl,
an aryl with multiple or fused rings in which at least one ring is aromatic, or
a heteroaryl.

4. The bleach catalyst according to claim 3, wherein R² is:
a branched alkyl or heteroalkyl having 4 to 16 carbon atoms, or
a cycloalkyl-, cycloalkenyl-, cycloalkynyl-, or aryl-substituted linear or branched alkyl having 2 to 16 carbon atoms in the alkyl chain;
then the carbon in R² that is bonded to the third carbon atom of the bleach catalyst having formula (1) is a quaternary carbon atom.

5. The bleach catalyst according to claim 3, wherein R² is:
a heterocyclic alkenyl,
a substituted aryl,
an aryl with multiple or fused rings in which at least one ring is aromatic, or
a heteroaryl.

6. The bleach catalyst according to claim 5, wherein R² is a heterocyclic alkenyl or a heteroaryl.

7. The bleach catalyst according to claim 6, wherein R² is pyridyl, quinolyl, isoquinolinyl, acridinyl, pyrazinyl, quinoxalinyl, imidazolyl, benzimidazolyl, purinyl, pyrazolyl, indazolyl, pyrimidinyl, quinazolinyl, pyridazinyl, or cinnolinyl.

8. The bleach catalyst according to claim 7, wherein R² is a pyridyl.

9. The bleach catalyst according to claim 1, wherein the C log P of the bleach catalyst is lower than 1.8.

10. A cleaning composition comprising the bleach catalyst according to claim 1, at least one bleaching agent, and at least one bleach activator.

11. The cleaning composition according to claim 10, wherein the composition is a hard-surface cleaning composition or a soft-surface cleaning composition.

12. The cleaning composition according to claim 10, wherein the composition comprises between 0.001 wt. % and 10 wt. % of the bleach catalyst, based on the total weight of the composition.

13. The cleaning composition according to claim 10, wherein the at least one bleaching agent comprises at least one source of active oxygen selected from the group consisting of an inorganic peroxide, an organic peracid, hydrogen peroxide, and combinations thereof.

14. The cleaning composition according to claim 10, wherein the at least one bleach activator is selected from the group consisting of tetraacetylethylenediamine (TAED), acetylated triazine derivatives, acetylated glycoluriles, acylimides, acetylated glucose, acetylated glycerol, acetylated phenolsulfonates, acetylated phenol carbonic acids, sodium nonanoyloxybenzenesulfonate (NOBS), 4-decanoyloxybenzoic Acid (DOBA), carbonic acid anhydrides, acetylated sugar derivatives, N-acetylated lactams, and combinations thereof.

15. A method of using the cleaning composition according to claim 10 for the removal of stains and/or for the removal of malodour from a garment, the method comprising: washing the garment with the cleaning composition of claim 10.

16. The method according to claim 15, wherein the garment is made of cotton, a synthetic material, or a combination thereof.

17. A method of washing clothes, wherein the method comprises providing the composition according to claim 10 to a laundry washing machine and performing the washing:
at a temperature lower than 60° C.; and
for a time of less than 120 minutes.

18. The method according to claim 15, wherein the cleaning composition according to claim 10 is used for the removal of stains comprising a polyphenolic compound.

19. The method according to claim 15, wherein the cleaning composition according to claim 10 is used for the removal of at least one of turmeric stains or curry stains.

20. The method according to claim 16, wherein the synthetic material is polyester.
